(19) United States
(12) Patent Application Publication
(10) Pub. No.: US 2011/0275936 A1
(43) Pub. Date: Nov. 10, 2011

(54) METHOD FOR DETERMINING SHEAR STRESS AND VISCOSITY DISTRIBUTION IN A BLOOD VESSEL

(76) Inventors: Daniel J. Cho, Wayne, PA (US); Seul Ki Jeong, Gwangju (KR)

(21) Appl. No.: 12/775,983

(22) Filed: May 7, 2010

(51) Int. Cl. A61B 8/00

(52) U.S. Cl. 600/438

(57) ABSTRACT

A method for computing patient specific blood viscosity and patient specific shear stress on a location of interest in the interior of a blood vessel that includes calculating shear rate of blood in the vessel, using imaging techniques and calculating the shear rate related blood viscosity.

### FIELD OF THE INVENTION

The present invention relates to a method for utilizing imaging in combination with the viscosity of blood to determine wall shear stresses and viscosity distributions at a selected locus inside a given blood vessel.

### BACKGROUND

Atherothrombosis and its associated clinical entities, including cardiovascular disease, stroke, and peripheral arterial disease, are leading causes of disability and mortality both in developed and developing countries.

The vascular system can generally be viewed as a closed circulatory system, constraining a series of biochemical processes and mechanical stresses to be controlled within the vessels for normal homeostasis. Thus, if mechanical stresses such as wall shear stresses and tensile stresses cannot be properly dispersed through vascular homeostasis, then one or more blood vessels may suffer injury. Through mechanotransduction, these stresses can trigger an inflammatory cascade, hyperplasia, and remodeling, resulting in atherosclerosis and subsequent thromboembolism.

In a normal, healthy blood vessel, a physiologic range of wall shear stresses are maintained by mechanical forces produced by blood flow. Wall shear stress in a blood vessel, i.e., the frictional force per unit area acting tangentially to the arterial wall, is determined by the product of shear rate (\dot{γ}) and blood viscosity (μ). The shear rate (\dot{γ}) is defined as the velocity gradient within the lumen and is determined by the first derivative of flow velocity with respect to the distance from the vessel wall. Viscosity (μ) is a fluid's resistance to flow.

Blood viscosity has long been reported to have an independent prognostic value for vascular diseases and, furthermore, is helpful in predicting conditions such as diabetes, hypertension, infections, and infarctions in addition to their complications.

Human blood is a fluid suspension of plasma and cells such as erythrocytes, leukocytes, and platelets. The viscosity of plasma, a Newtonian fluid, does not depend on characteristics of its flow. Whole blood, on the other hand, behaves as a non-Newtonian fluid, and its viscosity depends on its shear rate. Specifically, whole blood is more viscous at low shear rates and becomes relatively less viscous at higher shear rates. The shear rate dependent aspect of blood viscosity poses a challenge to accurately determining the wall shear stress in a specific blood vessel.

To date, all previous methods for determining wall shear stress have assumed blood to be a Newtonian fluid and its viscosity to be represented as a constant value. This is despite the important fact that whole blood viscosity varies, often widely, as a function of shear rate.

In U.S. patent application Ser. No. 12/668,270 a method is described that takes account of the shear-rate dependent viscosity of whole blood for determining the risk of rupture of a plaque formation in the interior of a blood vessel. Specifically, Ser. No. 12/668,270 discloses obtaining a profile of the plaque formation, and then using the profile so obtained to calculate the shear stress on the surface of the plaque formation along the axial direction of the blood flow, whereby regions of high shear stress can be identified. More specifically, the shear stress at each location is calculated using the diameter of the lumen at that location, and then the shear stress so calculated is compared to a threshold shear stress to determine whether any part of the plaque formation is at a risk of rupture. In addition to taking into account the change in the local diameter of the lumen to calculate shear stress at that location, Ser. No. 12/668,270 discloses that the viscosity of the blood is also taken into account.

Ser. No. 12/668,270 suggests using various blood vessel imaging methods to determine the diameter of the blood vessel including angiography, interferometric phase-contrast imaging technique, magnetic resonance imaging (MRI), three-dimensional MR angiography, CT, intravascular ultrasound, virtual arterial endoscopy, and endovascular probe, among others. Furthermore, Ser. No. 12/668,270 suggests measuring blood flow velocities using a wide variety of techniques including ultrasonic, radiographic, electromagnetic, pressure transducing, anemometric methods, among others, which can assist in the selection of shear rates used for viscosity measurement.

### BRIEF DESCRIPTION OF THE INVENTION

It is an object of the present invention to provide a more accurate measurement technique for wall shear stress that is specific to the flow characteristics of blood within the blood vessel and the viscosity of the blood. It is furthermore an object of the present invention to provide location specific viscosity distributions based on the flow characteristics within the blood vessel.

In a method according to the present invention shear rate (\dot{γ}) specific blood viscosity measurements are used to obtain more accurate artery specific shear stress measurements.

In accordance with one aspect of the present invention, a method is provided for computing wall shear stresses specific to flow conditions along a blood vessel. Specifically, the method provides for measuring the internal diameter of a blood vessel and the velocity of blood flow using a vascular imaging apparatus. The measurements can be taken over the duration of at least one cardiac cycle in order to determine the peak-systolic and end-diastolic diameters and velocities along the blood vessel. The values for systolic and diastolic shear rates can be obtained based on the peak-systolic and end-diastolic diameters and velocities measured through vascular imaging. The values so obtained can be then used along with shear rate specific viscosity values to obtain shear stress values for a locus of interest in the interior region of a blood vessel.

### BRIEF DESCRIPTION OF THE DRAWINGS

FIG. 1A depicts the interior region of a blood vessel having a plaque formation on a region of the interior thereof.

FIG. 1B shows a cross-sectional view of the vessel of FIG. 1A along line 1B-1B viewed in the direction of the arrows.

FIG. 2 illustrates a viscometer that can be used in a method according to the present invention.

FIGS. 3 and 4 show diffusion weighted brain MRI images for a subject studied using a method according to the present invention.

FIG. 5 illustrates carotid arteries of a first subject studied according to the present invention.

FIG. 6 illustrates carotid arteries of a second subject studied according to the present invention.

FIG. 7 shows a three-dimensional section of a stenosed artery.

FIG. 8 shows a simulation of wall shear stress on the interior wall of the section shown by FIG. 7.

FIG. 9 shows a map of patient specific viscosity data for the section shown in FIG. 7 according to another embodiment of the present invention.

FIG. 10 shows a map of patient specific shear stress data for the section shown in FIG. 7 according to another embodiment of the present invention.

FIG. 11 shows a cross-sectional view along line 11-11 in FIG. 9.

FIG. 12 is a flow chart showing the steps in a diagnostic application employing a method according to the present invention.

### DETAILED DESCRIPTION OF EMBODIMENTS OF THE INVENTION

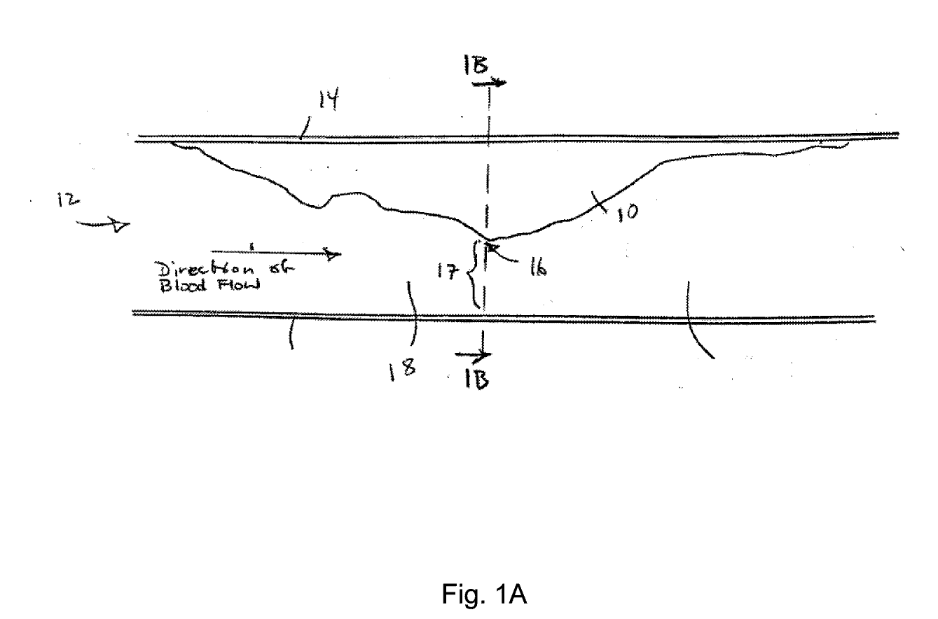

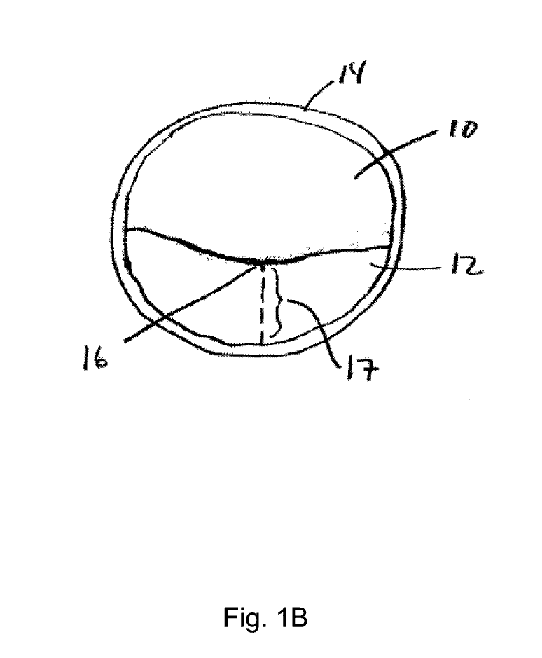

FIG. 1A illustrates a plaque formation 10 in the lumen 12 of a blood vessel 14 (e.g. an artery) of a human being. As discussed, U.S. patent application Ser. No. 12/668,270 discloses that the change in the viscosity of blood at various shear rates may increase the potential for the rupture of plaque formation 10 by increasing the shear stress thereon. Another factor that may increase shear stress on the rupture of a plaque formation is the reduction in the dimension of lumen 12. Specifically, the reduction in the lumen increases the shear rate of blood thereby increasing shear stress on the plaque formation. Thus, for example, at the point of maximum blockage 16, where lumen 12 is narrowest, the shear rate of blood may increase, thereby increasing shear stress on plaque formation 10 and consequently increasing the chances of plaque rupture.

A method according to the present invention can be used to determine a value for the shear stress at a locus in an interior region of a blood vessel 14. For example, at a locus of interest, shear stress caused by blood flow on a plaque formation 10, such as the point of maximum blockage 16, can be calculated.

According to one aspect of the present invention, an electronic imaging device, such as a duplex ultrasonograph, can be used to obtain an image of the interior of a blood vessel 14 at a desired instant in time. Naturally, the image so obtained would include the image of the locus of interest. For example, the image would include the portion of an artery having a plaque formation on the interior wall thereof. The instant in time can be selected to correspond to peak systole (high shear rate), or end diastole (low shear rate) for which approximate shear rate of blood is known. For example, it is known that at high systole shear rate of blood is approximately 300 s⁻¹ and at end diastole the shear rate of blood is approximately 5 s⁻¹. The speed of blood passing the locus of interest at the selected instant of time is then obtained. For example, the speed of blood passing position 16 (narrowest region of lumen 12) is determined using duplex ultrasonography at peak systole or end diastole. According to one aspect of the present invention the value of the speed is measured at the center of the blood stream passing the locus of interest, which would correspond to the maximum speed of blood passing the locus of interest.

Thereafter, an interior dimension of the blood vessel, such as the diameter 17 of lumen 12 at position 16 can be measured using the image. Referring to FIGS. 1A and 1B, it should be understood that diameter as referred to herein does not necessarily refer to the diameter of a circle. Rather, diameter 17 refers to the distance between the locus of interest and the directly opposite region on the interior surface of the vessel, which should be the shortest distance between the two regions. With values of speed and dimension, shear rate at high systole and end diastole can be obtained. These values can be then used to obtain shear stress at the locus of interest at high systole and end diastole by measuring blood viscosity at a shear rate corresponding to the high systole and end diastole.

Thus, the value of the viscosity of the blood flowing through lumen 12 can be measured at a number of shear rates, and the viscosity at the shear rate corresponding most closely to the speed value can be used to calculate shear stress of the blood at the locus of interest. For example, if the speed is obtained at high systole, a blood viscosity value at high shear rate (e.g. approximately 300 s⁻¹) may be used, or if the blood speed is measured at end diastole then a viscosity value at low shear rate (e.g. approximately 5 s⁻¹) may be used. Thereafter, by multiplying the shear rate value and the viscosity value at the instant in time, the shear stress value at that instant in time can be obtained for the locus of interest. For example, the shear rate value at high systole can be multiplied by the viscosity at high shear rate to obtain a shear stress at high systole at the locus of interest, or the shear rate value at end diastole can be multiplied by the viscosity value at low shear rate to obtain shear stress at the locus of interest at end diastole. Thus, shear stress can be calculated at a locus of interest inside the lumen of a blood vessel at one instant, e.g. high systole, and at another instant, e.g. low diastole, using the methodology set forth above.

It should be noted that the steps of obtaining the maximum speed of blood, the lumen diameter, and blood viscosity need not be taken in any particular order.

In one embodiment, duplex ultrasound can be used to capture and store a video image of the interfaces between lumen and intima of a blood vessel of a person for the duration of a cardiac cycle encompassing peak-systole and end-diastole. For example, video images of a portion of the carotid arterial segment, such as the common carotid artery, carotid bulb, or internal carotid artery can be obtained and used to measure the internal diameter at a locus of interest and maximum centerline velocity of the blood passing the locus of interest. Intra-luminal diameters can be measured separately at peak-systole (at the R wave) and end-diastole (at the T wave). The diameter of the lumen corresponding to an exact instant in time, such as the R wave or T wave, can be measured by freezing the video images.

In this embodiment, the blood flow velocities can be measured using duplex ultrasonography at both peak-systole and end-diastole with the sample volume reduced to a small size and placed in the center of flow, thereby enabling the maximum centerline velocity to be determined. The Doppler angle may be maintained at 45°±4 without exceeding 60°. The sample volume box may be placed in the mid-lumen parallel to the vessel wall for a relatively healthy artery, or the sample volume box may be aligned parallel to the direction of blood flow in the case of a significantly diseased artery with atherosclerotic plaques.

In this embodiment, the systolic and diastolic shear rates can be determined using the following relationship:

$$\dot{γ} = \frac{4 V}{D}$$

where V and D are the maximum centerline velocity and lumen diameter at the locus of interest, respectively.

To obtain the blood viscosity values at high shear rate and low shear rate, a computerized scanning capillary viscometer can be used. An example of such a system that uses the Casson equation is disclosed in U.S. Pat. Nos. 6,796,168 B1 Goldstein et al. (incorporated by this reference), and 6,745,615 B2 Kensey et al. (incorporated by this reference). The Casson equation provides a relationship between the shear stress (τ) and shear rate (\dot{γ}). The Casson equation is given as:

\sqrt{τ}=\sqrt{τ_{y}}+\sqrt{k\dot{γ}} for τ>τ_{y}

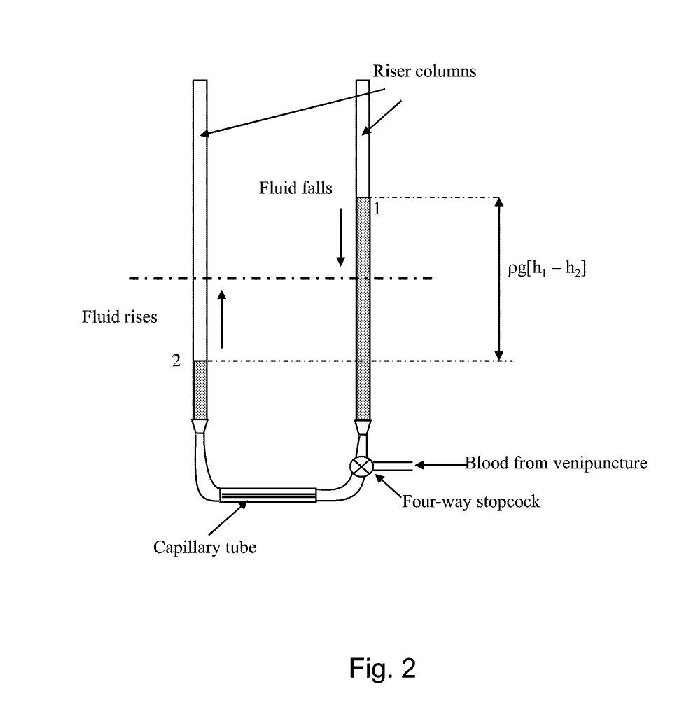

\dot{γ}=0 for τ≦τ_{y}

where τ_{y} is the first Casson model constant and is interpreted as the yield stress, and k is a second Casson model constant, which reflects the asymptotic value of blood viscosity at a high shear rate. The computerized scanning capillary viscometer referred to above can calculate the wall shear stress and shear rate from flow velocity and pressure drop measurements in a capillary tube. Qualitatively, the fluid dynamic theory used in the scanning capillary viscometer is similar to the Poiseuille flow relation, which describes blood viscosity (μ) as:

$$μ = \frac{π {d}^{4} Δ P}{128 QL}$$

where Q is the volume flow rate of blood, d is the inside diameter of a capillary tube, ΔP is the pressure drop along the capillary tube length, μ is blood viscosity, and L is the length of the tube. The computerized scanning capillary viscometer used in this embodiment consists of two main components: a height detection system to measure height variations in the two riser columns and a U-shaped disposable tube. FIG. 2 shows a sketch of the U-shaped tube unit comprised of two vertical riser columns connected by a horizontal capillary tube. Blood is first introduced into the right riser column through a stopcock valve. Once the right column is filled with blood, the blood is then introduced into the left column using the computer-controlled three-way stopcock, allowing the blood in the right side to fall and the blood in the left side to rise. By design, the right and left fluid levels come to equilibrium or to an asymptotic point as indicated by the thick dashed line in FIG. 2. The pressure drop is determined from the height difference measurement (i.e., (ρg[h₁−h₂]) in the two riser columns, while the volume flow rate Q(t) is mathematically determined using the first derivative of the height with respect to time, dh/dt. Since the diameter and length of the capillary tube are known values, one can determine the blood viscosity from the pressure drop and flow rate data. Note that the geometry of the U-tube controls the flow rate, and thus, the shear rate, at the capillary tube from an initial maximum value to almost zero as the two fluid levels in the riser columns approach each other. Therefore, the blood viscosity values measured can be obtained over a wide range of shear rates, i.e., from 1000 to 1 s⁻¹.

The blood viscosity values computed by a scanning capillary viscometer that correspond to the systolic and diastolic shear rates in a given vascular segment are specified using the two Casson model constants (k and τ_{y}):

$$\sqrt{τ} = \sqrt{{τ}_{y}} + \sqrt{k \dot{γ}}$$

$$W B V ( μ ) = \frac{τ}{\dot{γ}} = \frac{{τ}_{y}}{\dot{γ}} + k + 2 \sqrt{\frac{k \cdot {τ}_{y}}{\dot{γ}}}$$

According to one aspect of the present invention, wall shear stress values are computed correspondingly, on one hand, to the systolic and diastolic shear rates produced by the vascular imaging modality, and on the other hand, to the systolic and diastolic blood viscosities (i.e., μ_{S} and μ_{D}) that are themselves corresponding to subject-specific and vessel-specific systolic and diastolic shear rates. Specifically, using the vascular imaging modality the diameter of the locus of interest and the maximum speed of blood at a particular time of interest are obtained from which the shear rate specific to the locus and time of interest (patient specific shear rate) can be determined. The scanning capillary viscometer is then used to obtain the necessary information for the calculation of the shear stress and viscosity at the locus and time of interest. For example, Casson model constants can be obtained from the measurements performed by the viscometer. Then, the information obtained from the viscometer can be used along with the shear rate calculated at the locus and time of interest to obtain the viscosity of blood at the locus and time of interest (patient specific blood viscosity), and shear stress at the locus and the time of interest (patient specific shear stress). Thus, for example, Casson model constants obtained through blood viscometry are used to calculate blood viscosity and blood shear stress at the locus and time of interest. The systolic wall shear stress and the diastolic wall shear stress are calculated as follows:

Systolic shear stress (τ_{S})=\dot{γ}_{S}×μ_{S}

Diastolic shear stress (τ_{D})=\dot{γ}_{D}×μ_{D}

It should be noted that a method according to the present invention computes wall shear stress values by taking into account non-Newtonian characteristics of blood. That is, a method according to the present invention takes account of the fact that blood viscosity varies as a function of shear rate. Numerous methods have been previously described for calculating the wall shear stress based on the assumption that blood viscosity is a constant and does not vary with its flow rate. However, such an assumption leads to inaccurate shear stress calculations and thus inaccurate analyses of the condition surrounding the rupture of plaque formations.

A method according to the present invention can be used to determine shear stress at a location of interest in an interior region of a blood vessel at high systole and at end diastole. Thus, according to one aspect of the present invention, the difference between systolic shear stress and diastolic shear stress, designated as pulse shear stress (PSS=τ_{S}−τ_{D}), can be calculated and used for diagnostic purposes as illustrated below.

According to another aspect of the present invention, the difference between systolic blood viscosity and diastolic blood viscosity, designated as pulse blood viscosity (PBV=μ_{D}−μ_{S}), can be used for diagnostic purposes as illustrated below.

The following discloses a feasibility study in which a method according to the present invention is employed to obtain diagnostic information. The study was approved by the relevant institutional ethics committee and involved two human subjects (Subject 1 and Subject 2). The study was carried out after obtaining written, informed consent from the subjects.

Subject 1 was diagnosed with cerebral infarction (stroke), and Subject 2 with transient cerebral ischemic attack (TIA). The diagnosis of cerebral infarction was made following a history of ischemic stroke with evidence of acute focal neurological dysfunction and symptoms lasting more than 24 hours. These symptoms were viewed to be a result of intracerebral ischemia on the basis of diffusion-weighted brain magnetic resonance imaging. Subject 2 presented with brief episodic neurological dysfunction caused by a focal disturbance of brain ischemia with clinical symptoms typically lasting less than 1 hour and without evidence of infarction, and Subject 2 was diagnosed with TIA.

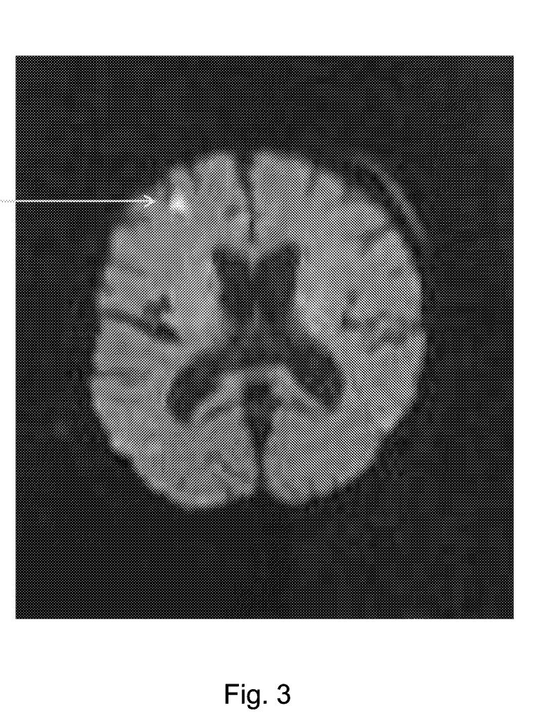

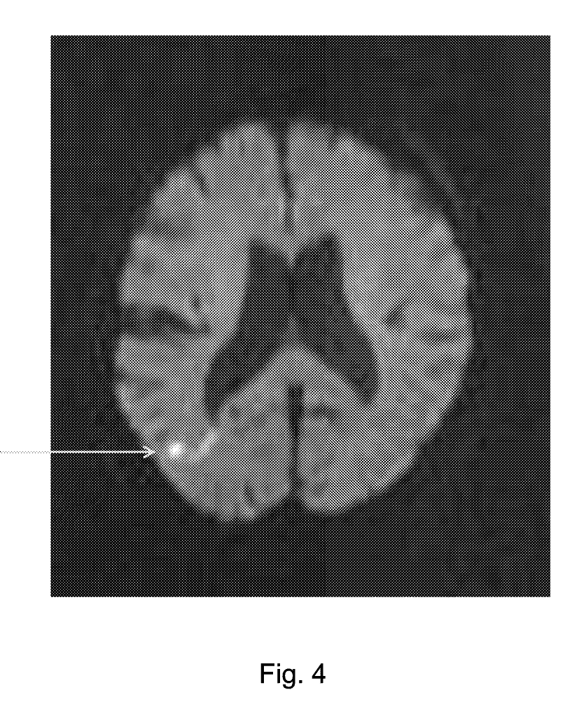

Subject 1 with a cerebral infarction was a 78-year-old male and exhibited left-sided hemiparesis (MRC grade IV for the upper extremity and V⁻ for the lower extremity), left central type facial palsy, hemihypesthesia, and dysarthria. Diffusion-weighted brain MRI showed high signal intensity lesions in the right-sided anterior and posterior watershed areas, as shown in FIGS. 3 and 4 (identified by an arrow in each figure). Subject 1 engaged in heavy alcohol consumption and smoking of more than 60 pack-years. Additionally, Subject 1 had dyslipidemia and elevated plasma concentration of tHcy (27.51 μmol/L). More than 10 years ago, Subject 1 had been diagnosed with thyroid cancer and had undergone thyroidectomy and radiotherapy. Subject 2 with TIA was a 56-year-old male and exhibited left-sided transient hemiparesis. Brain MRI did not reveal any acute lesions. With regard to risk factors, Subject 2 had hypertension and dyslipidemia.

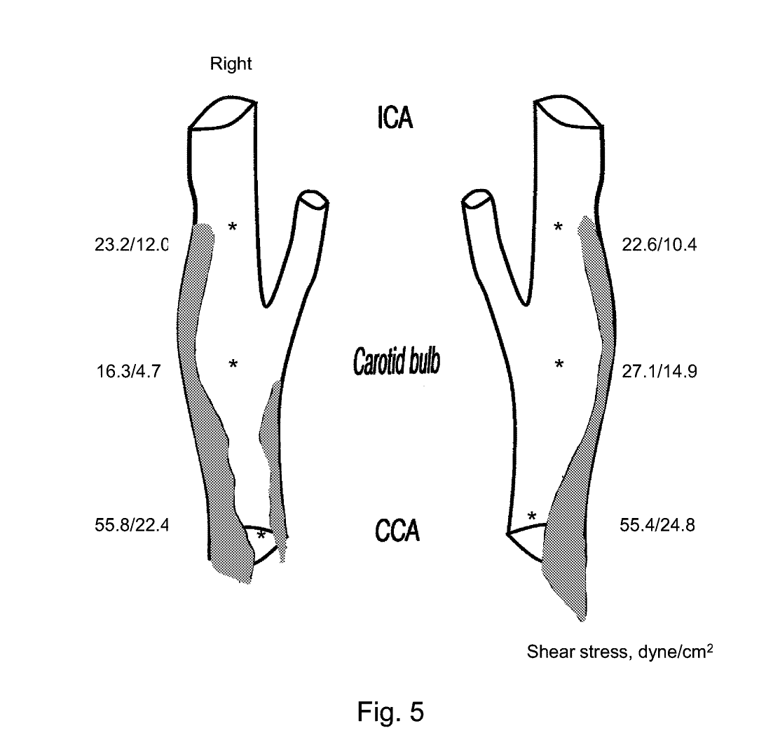

The two subjects were further examined using carotid duplex ultrasonography and blood viscometry according to the embodiment detailed above. The blood viscosity of Subject 1 with the watershed cerebral infarction was observed to be 4.88 cP (mPa·sec) at a shear rate of 300 s⁻¹ (high shear rate corresponding to peak systole) and 13.13 cP at a shear rate of 5 s⁻¹ (low shear rate corresponding to end diastole). All values for blood viscosity from shear rates between 1 and 1000 s⁻¹ were recorded simultaneously using a computerized scanning capillary viscometer referred to above. It is noted that values for normal blood viscosity at the high and low shear rates are 3.9 and 9.0 cP, respectively. Carotid ultrasonogram showed extensive and confluent echogenic heterogeneous plaques at both the right and left sides, as shown in FIG. 5. The narrowest peak-systolic/end-diastolic diameters at the right and left common carotid arteries (CCAs) were 0.24/0.22 and 0.37/0.34 cm, respectively, as shown in Table 1. At the right and left common carotid artery points of maximum stenoses, the systolic/diastolic values for blood viscosity—as determined on a shear rate specific basis—were found to be 4.43/4.70 cP and 4.43/4.66 cP, respectively. These were among the lowest values for systolic and diastolic viscosities observed in this mechanistic feasibility study. Additionally, systolic/diastolic shear stresses—again, determined on a shear rate specific basis and, additionally, using the corresponding blood viscosity values—were highest at the narrowest points of the both right and left CCAs, which were 55.8/22.4 dyne/cm² and 55.4/24.8 dyne/cm², respectively.

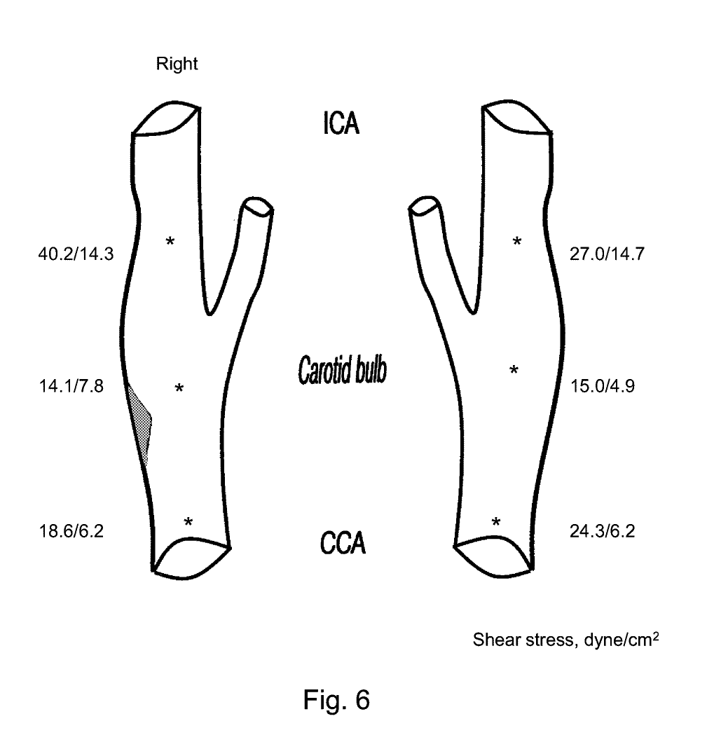

The blood viscosity of Subject 2 with TIA was observed to be 3.79 cP at shear rate of 300 s⁻¹ and 9.24 cP at a shear rate of 5 s⁻¹, which happened to be lower than those of Subject 1 with the cerebral infarction. Carotid ultrasonogram showed a small-sized echogenic homogenous plaque in the right CCA to carotid bulb transition area, as shown in FIG. 6. The narrowest peak-systolic/end-diastolic diameters at the right and left-sided CCAs were 0.79/0.69 cm and 0.73/0.64 cm, respectively, as shown in Table 1. The shear rate specific systolic/diastolic blood viscosities at the right and left CCA segments, were 3.67/4.07 cP and 3.61/4.07 cP, respectively. The highest shear rate-specific shear stresses were observed in ICAs for both the right and left sides.

The detection of sudden changes in systolic or diastolic shear stresses along the enthothelial lining for the purpose of monitoring and predicting the progression of atherosclerosis is an additional aspect of the present technique. The right carotid bulb in Subject 1 with cerebral infarction (FIG. 4) showed sudden decreases in systolic wall shear stresses from 55.8 dyne/cm² to 16.3 dyne/cm², and the diastolic wall shear stress from 22.4 dyne/cm² to 4.7 dyne/cm², resulting in net decreases of 39.5 dyne/cm² and 17.7 dyne/cm² in systolic and diastolic shear stresses, respectively, along the arterial segments. The lateral wall of the poststenotic dilated arterial segments—with sudden changes in the measured centerline shear stresses—would face stresses in the diastolic (or low shear) regime because of recirculating eddy flow in that segment. As such, such segments could be sites prone to future plaque growth.

In Subject 1, pulse blood viscosity (PBV) ranged from 0.23 (left CCA) to 0.95 cP (right carotid bulb), and pulse shear stress (PSS) ranged from 11.2 (right ICA) to 33.4 dyne/cm² (right CCA). In Subject 2, PBV ranged from 0.15 (left ICA) to 0.47 cP (left carotid bulb), and PSS ranged from 6.3 (right carotid bulb) to 25.9 dyne/cm² (right ICA).

The data reported above demonstrates the feasibility of a method according to the invention for computing blood viscosity and shear stress on a shear rate specific basis at the bifurcation site of the carotid artery. As will be appreciated by one who is skilled in the art, this method is not limited to application at the carotid artery or cerebrovasculature, but to any blood vessel of the body.

To compute the viscosity values for the study Casson model constants (k and τ_{y}) were derived from blood viscosity data obtained from a computerized scanning capillary viscometer. However, as should be evident to one skilled in the art, any viscometer or rheometer capable of generating viscosity values across a range of shear rates could serve in this capacity.

As is demonstrated by the feasibility data, shear stress behaves as a dynamic parameter, and its range is widely variable along a segment of a blood vessel—in this case, along the carotid arterial lining from CCA to internal carotid artery (ICA). Using this methodology, the differences between systolic and diastolic values for both blood viscosity and shear stress could be determined. These differences are an additional aspect of the present invention and are designated herein as pulse blood viscosity (PBV) and pulse shear stress (PSS). The loci of the largest differences (i.e., the largest PBV and PSS values) could be detected relatively easily using the present method.

According to another aspect of the present invention, PSS can be sued as a predictor of plaque vulnerability. In Subject 1 with right-sided, middle carotid artery (MCA) territorial watershed cerebral infarction, the systolic shear stresses of both common carotid arteries were nearly same, but PSS was higher in the right, as shown in FIG. 5. In Subject 2 with a left-sided transient hemiparesis, the right-sided internal carotid artery showed the highest systolic shear stress and highest PSS as well. It is noted that the present method does not reveal the etiopathogenic aspects of cerebral ischemia of the two patients, i.e., whether they were due to artery-to-artery embolism or hemodynamic derangement (low flow); with the exception of the lesion in the carotid arteries, neither patient displayed any other measurable atherosclerotic lesions in the cerebral vasculature.

In the feasibility study, the strongest shear stresses were observed to occur in the most stenotic segment of the artery (i.e., the segment having the smallest diameter), as shown in Table 1. Furthermore, the weakest shear stresses were observed at the segments with the largest opening such as the post-stenotic dilated segment or carotid bulb. Values for blood viscosity were lowest at the most stenotic segments and highest in the arterial segments with the largest opening. In contrast, if blood was assumed to behave as a Newtonian fluid, and a certain representative value was used, as is presently practiced and published throughout the field, wall shear stresses would be calculated as higher than their true values in the stenotic segments, and lower in the dilated segments. This discrepancy would become more pronounced in severely pathologic arterial segments, i.e., where accuracy is most needed. These insights are generated by the present methods and are underscored by the non-Newtonian behavior of blood flow.

Many previously described or published methods to calculate or measure arterial shear rates use vessel cross-sectional area rather than vessel diameter. The present method is not limited by the use of diameter as opposed to cross-sectional area, and it will be appreciated to one skilled in the art that the second power of the vessel diameter may be used to determine vessel cross-sectional area to calculate shear rate. An important aspect of the present method is the integration of such shear rate values, as produced by vascular imaging means, together with values for blood viscosity determined on a shear rate specific basis, in order to generate measurements for wall shear stress which themselves correspond to shear rates and blood viscosities that are subject-specific and artery-specific, and additionally, that such a methodology shall produce wall shear stress measurements that more precisely describe actual physiological conditions.

It has been previously described and published that a lesion facing shear stresses lower than the normal physiological value of 10-15 dyne/cm² could be prone to atherosclerotic growth and that the plaque segment facing a shear stress higher than the normal range could be vulnerable to rupture (or plaque instability). It is understood that wall shear stress in an artery is homeostatically maintained within a physiological range by means of normal vascular function, i.e., mechanisms for vasodilation and vasoconstriction, and that normal physiological wall shear stress levels in turn serve to maintain vascular tone through the mediation of nitric oxide. However, in a diseased artery with significant stenosis, the wall shear stress can be significantly greater or smaller than the values in the normal physiological range, particularly at the proximal and distal regions of the stenosis, respectively. A method according to the present invention factors in the shear rate dependent viscosity to determine the wall shear stress more precisely for the purpose of enabling improved prediction of subsequent atherosclerotic progression or plaque vulnerability.

In the study disclosed herein, the shear rate-specific blood viscosity was calculated using the two Casson model constants together with the flow information obtained from ultrasonography. It should be understood that a method according to the present invention is limited neither to the shear-viscosity computations performed using the Casson model, nor to vascular imaging performed using duplex ultrasonography. Other viscometry models such as the Herschel-Buckley, Cross, and power-law models, among others, may be used. Other means for vascular imaging such as angiography, interferometric phase-contrast imaging, magnetic resonance imaging, three-dimensional MR angiography, computed tomography (CT), intravascular ultrasound, virtual arterial endoscopy, and endovascular probes, among others, may be used.

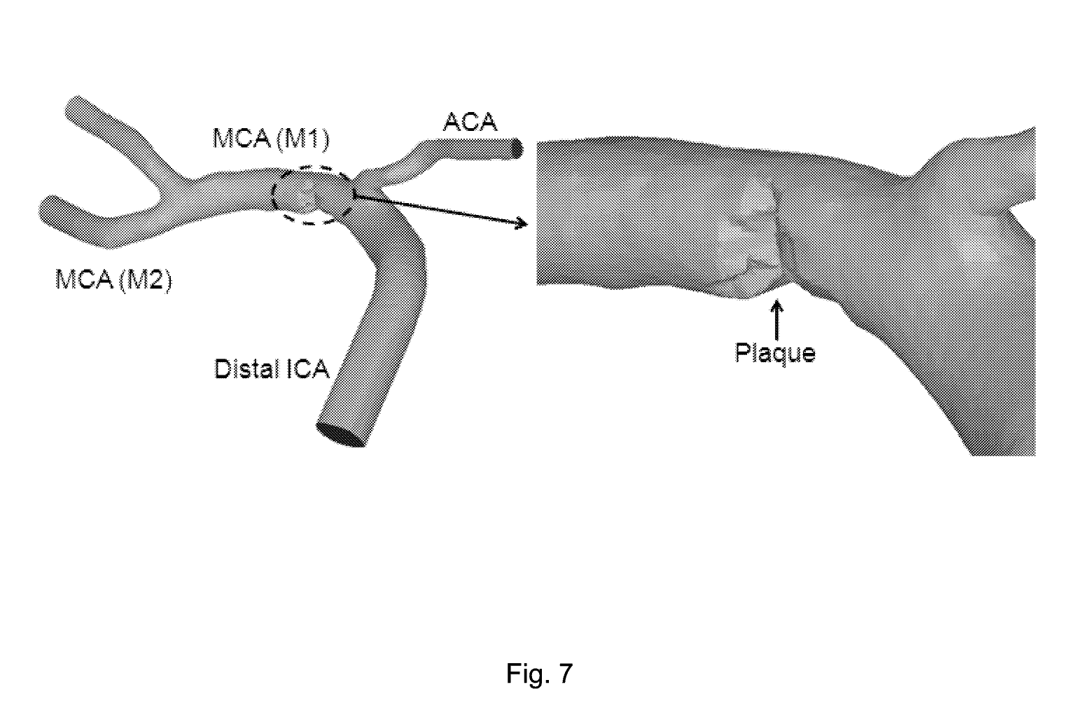

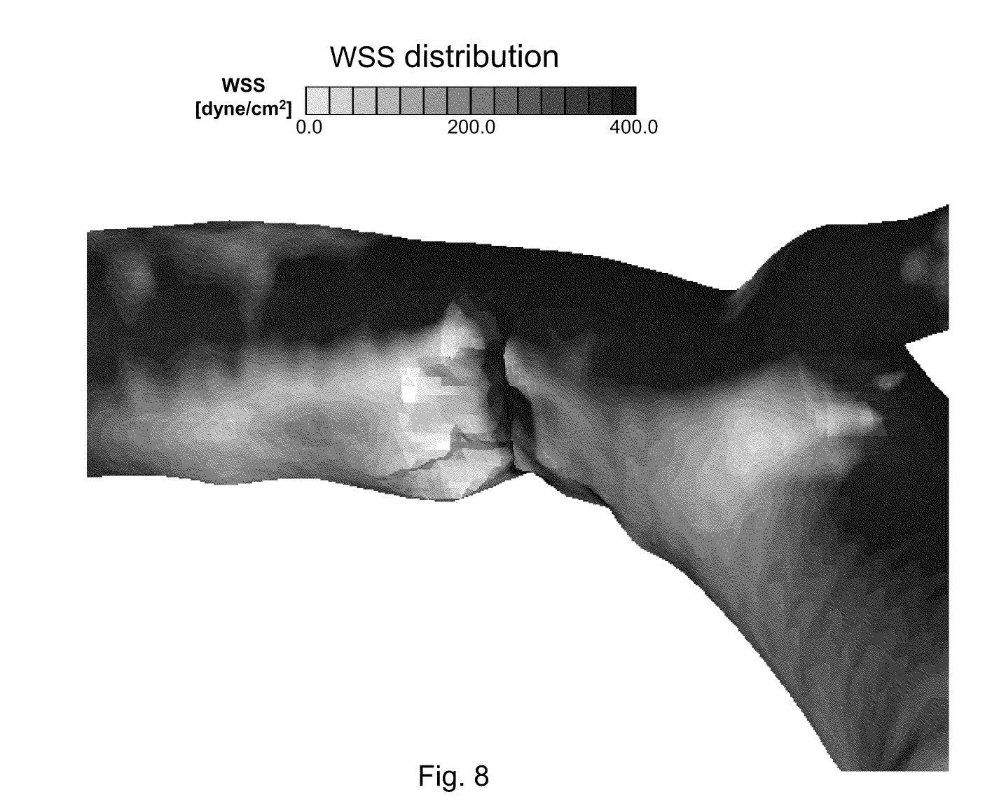

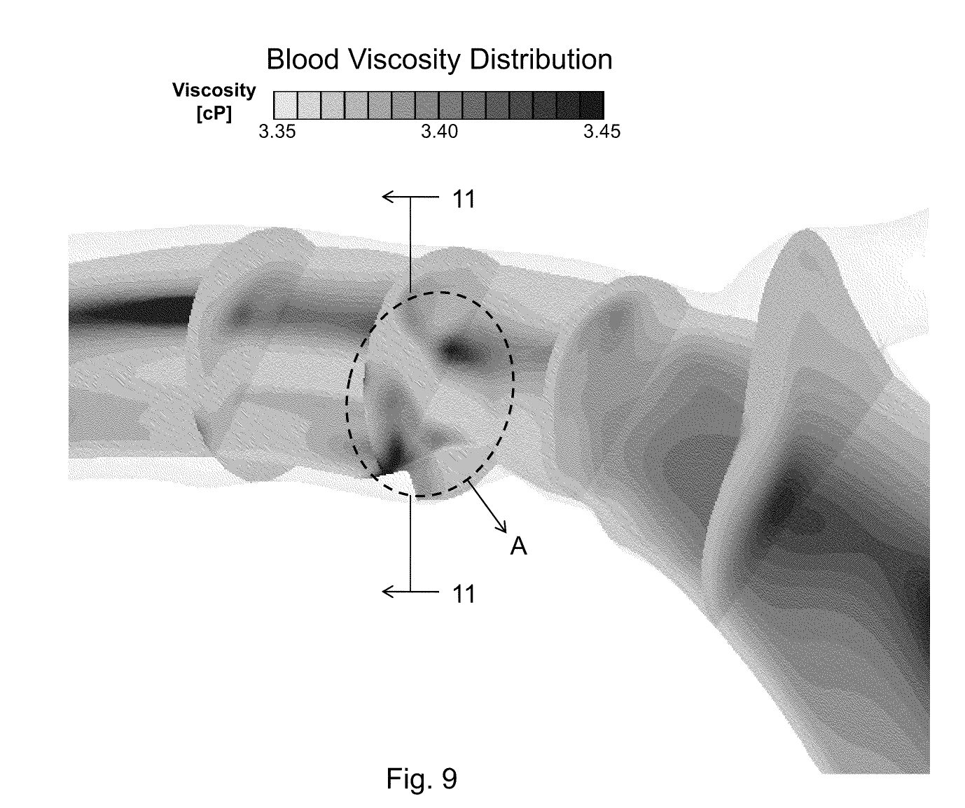

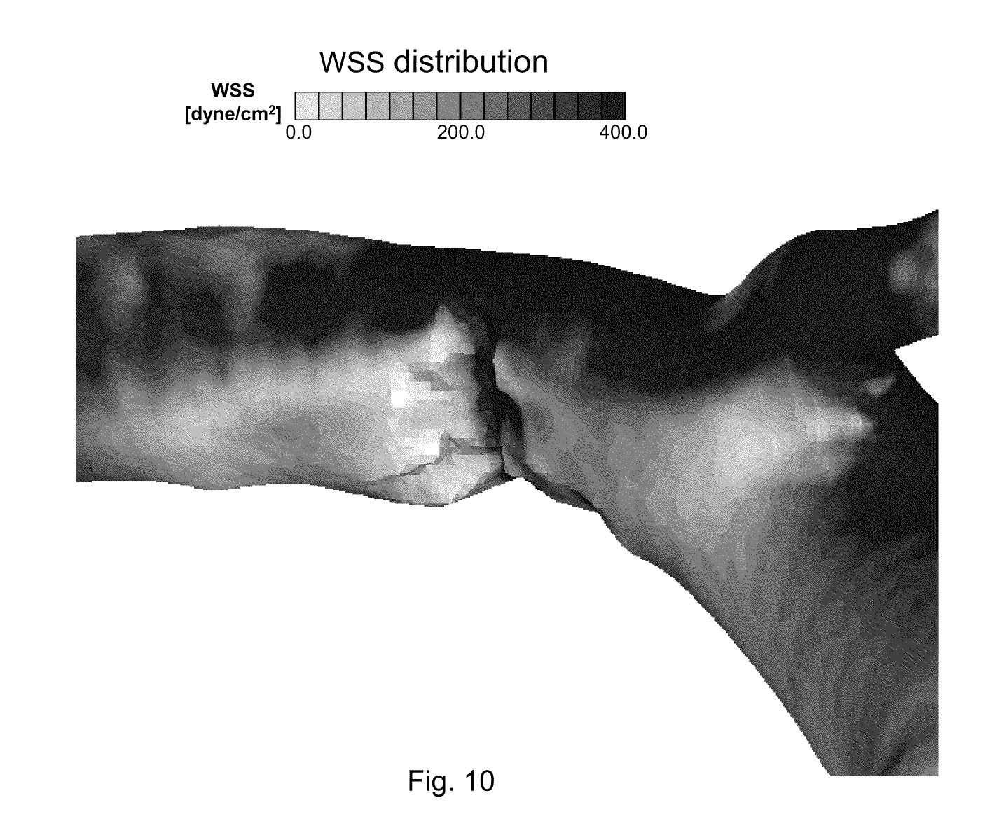

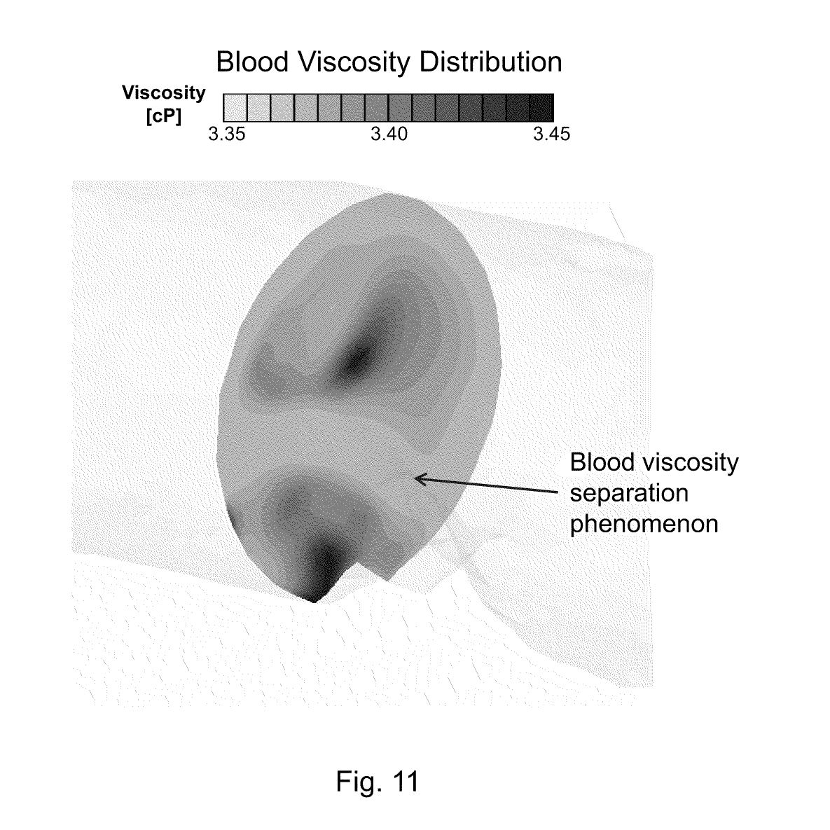

As another aspect of the present invention, a method according to the present invention may be employed to simulate blood flow characteristics computationally along an entire region of a blood vessel using patient-specific arterial geometry. For example, three-dimensional (3D) geometry of a carotid artery can be constructed from 2D brain magnetic resonance angiography (MRA) data of a person, called Subject 3. FIG. 7 illustrates a three dimensional image of a stenosed portion of the carotid artery of Subject 3. With the 3D reconstructed arterial geometry, the corresponding computational meshes can be generated using standard computational fluid dynamic techniques. For example, a software called Computational Fluid Dynamics can be used. Boundary conditions for the simulation of blood flow include patient-specific blood velocities obtained using transcranial doppler (TCD) examinations. Blood viscosity can be experimentally measured (for said Subject 3) over a comprehensive range of shear rates, for example, shear rates encompassing all or a portion of the shear rates from peak systole and end diastole. Thereafter, patient specific blood viscosity and patient specific wall shear stress (WSS) at any locus of interest can calculated for the selected portion of the artery and mapped preferably three-dimensionally for better visual analysis, although other mapping techniques can be used without deviating from the scope and spirit of the present invention. The blood velocity used was measured using Transcranial Doppler, which obtained the following values: V_{ICA}=86.6 cm/s (orbital window), V_{ACA}=84.3 cm/s (temporal window). Patient specific viscosity was calculated from the following measured blood viscosity values: constant viscosity at 300 s⁻¹=3.99 cP, and shear rate specific viscosity values were based on the measured Casson model constants τ_{y}=8.978E-2 dyne/cm², k=3.344E-2 dyne.s/cm². FIG. 8 shows a simulation of wall shear stress in the selected portion of the artery shown in FIG. 7. The mapped data are based on a shear rate of 300 s⁻¹ and a constant blood viscosity of 4 cP. FIG. 9 shows a map of data for the viscosity of the blood of subject 3 for the selected region shown by FIG. 7, the data being calculated according to the present invention. FIG. 10 shows a map of data for the shear stress caused by the blood of subject 3 on the wall of the selected region shown by FIG. 7, the data being calculated according to the method disclosed herein. FIGS. 7-10 demonstrate that when the non-Newtonian characteristics of whole blood are taken into account, the effect of shear rate thereof along any selected portion of an artery or other blood vessel can be better understood. For example, a comparison of FIGS. 7 and 10 indicates that patient specific wall shear stress data generates a substantially similar shear stress distribution pattern as when blood viscosity is assumed to be very high (4 cP) and having a high shear rate of 300 s⁻¹ although at that high shear rate the patient's blood viscosity should be well below 3.45 cp. FIG. 9 reveals that blood viscosity is stratified when calculated according to the present invention. The stratification pattern is helpful in identifying areas within the stenosed region that may require treatment. For example, the mapped data illustrates that there is a sudden jump in the blood viscosity value with respect to an adjacent value, within the post-plaque dilated region A. This phenomenon occurs in the region of the low wall shear stress. This phenomenon can be termed “blood viscosity separation” for the purposes of this invention because normal laminar flow with continuous gradients in blood viscosity has been separated into regions of relatively high blood viscosity linked by regions of relatively low blood viscosity. That is, instead of gradual change in viscosity, an abrupt change is observed. Other terminology may be used to describe this blood viscosity separation. FIG. 11, which shows a cross-sectional view of the mapped data of region A illustrates this phenomenon graphically. Specifically, the mapped data in region A indicates that blood is virtually motionless, which could be due to generation of an eddy current in the region after the stenosed region of the artery.

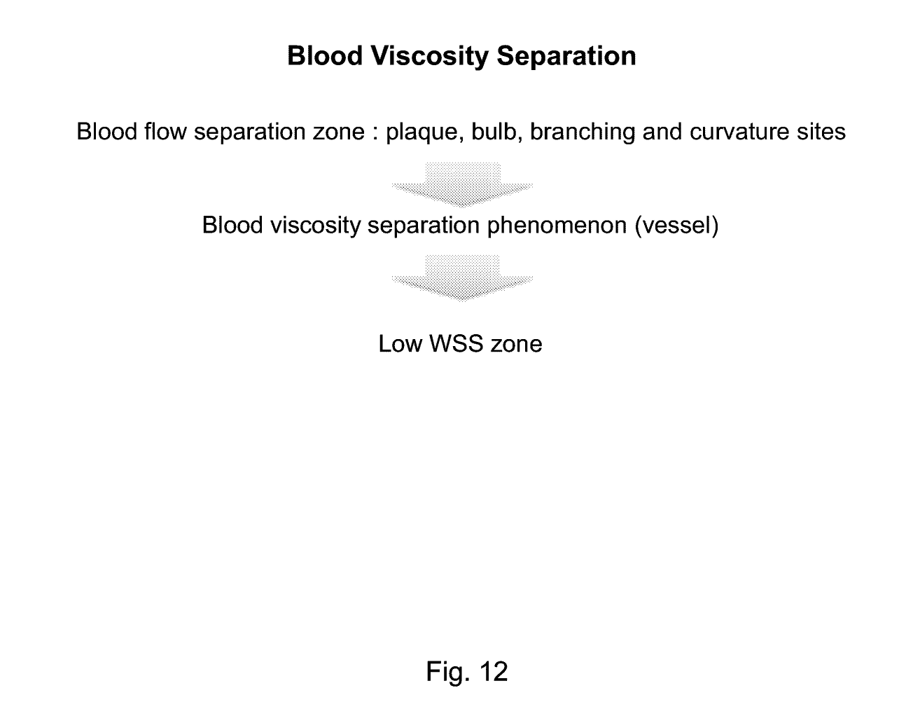

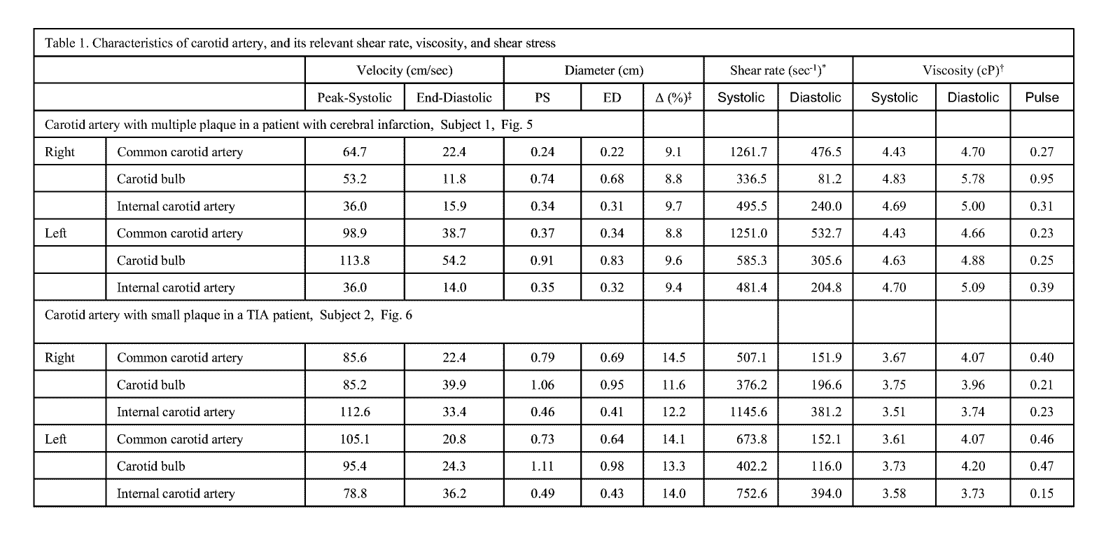

The identification of blood viscosity separation and the high viscosity surges in the post-stenotic dilated region of the artery may suggest active interaction of blood components with the endothelial cells and is suggestive of future growth of plaque. Blood viscosity separation and high viscosity surge, therefore, can represent clear diagnostic insights that are observable, targetable, druggable, and treatable by clinicians. Thus, according to another aspect of the present invention patient specific blood viscosity calculation as disclosed herein can be used to identify regions within a blood vessel in need of treatment. FIG. 12 is provided as a schematic of the flow of logic from the separation of blood flow created by vessel branches to the identification of increased risk of plaque growth. Note that the mapped data may not only suggest a location prone to further plaque growth, it may also be used to identify a location prone to plaque rupture.

The blood viscosity can be measured using any method including tube-type viscometers, rotational viscometers, microfluidic channel-type viscometers, porous bed viscometers, ultrasonographic viscometers, catheter-type viscometers, and other functionally similar instruments, so long as the measured viscosity values span a range of shear rates for the given blood specimen. That is any viscometer, which can produce a range of values either simultaneously or serially, may be used to provide viscosity measurements in the present invention will be evident to a person skilled in the art.

Importantly, although the examples provided herein were drawn from the cerebrovasculature, as will be readily apparent to a person skilled in the art, any blood vessel may be used to generate the required data. A key example of such other blood vessels would be the coronary arteries.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

What is claimed is:

1. A method to determine a value for patient specific blood characteristics at a locus in an interior region of a blood vessel, comprising:
obtaining an electronic image of said blood vessel at an instant, said image including said locus on said interior region of said blood vessel;
obtaining a speed value indicating speed of blood passing said locus on said interior region of said blood vessel at said instant;
measuring an interior dimension of said blood vessel at said locus based on said electronic image; and
determining a value for shear rate of said blood corresponding to said speed value and said interior dimension.

2. The method of claim 1, wherein said instant corresponds to peak systole.

3. The method of claim 1, wherein said instant corresponds to end diastole.

4. The method of claim 1, wherein said interior dimension is a diameter of said blood vessel at said locus.

5. The method of claim 1, wherein said speed value is a maximum velocity of blood.

6. The method of claim 1, wherein said electronic image is obtained by duplex ultrasonography.

7. The method of claim 1, wherein said speed value is obtained by duplex ultrasonography.

8. The method of claim 1, further comprising calculating patient specific blood viscosity at said locus based on said speed value, said dimension of said blood vessel at said locus based on said electronic image, and a measured value for viscosity of said blood.

9. The method of claim 1, further comprising calculating shear stress at said locus based on said speed value, said dimension of said blood vessel at said locus based on said electronic image, and a measured value for viscosity of said blood.

10. The method of claim 1, further comprising obtaining an electronic image of said blood vessel at another instant, said image including said locus on said interior region of said blood vessel;
obtaining another speed value indicating speed of blood passing said locus on said interior region of said blood vessel at said another instant;
measuring an interior dimension of said blood vessel at said locus based on said another electronic image;
determining a value for shear rate of said blood corresponding to said another speed value.

11. The method of claim 10, wherein said instant corresponds to peak systole and said another instant corresponds to end diastole.

12. A method wherein said steps in claim 1 are repeated for different locations along at least a portion of said vessel to obtain a wall shear stress value for all said locations, and further comprising mapping said wall shear stress values along said portion of said vessel.

13. A method wherein said steps in claim 1 are repeated for different locations along at least a portion of said vessel to obtain a blood viscosity value for all said locations, and further comprising mapping said blood viscosity values along said portion of said vessel.

14. A method according to claim 13, further comprising identifying locations that may be prone to plaque growth based on said mapped values.

15. A method according to claim 13, further comprising identifying locations that may be prone to plaque rupture.

16. The method of claim 1, wherein said electronic image is two-dimensional.

17. The method of claim 1, wherein said electronic image is three-dimensional.
